(19) United States
(12) Patent Application Publication
(10) Pub. No.: US 2012/0087985 A1
(43) Pub. Date: Apr. 12, 2012

(54) SMALL PEPTIDE SEQUENCES FOR STABILIZING BIOMOLECULES

(75) Inventor: Solomon S. Steiner, Mount Kisco, NY (US)

(73) Assignee: Biodel Inc.

(21) Appl. No.: 13/253,410

(22) Filed: Oct. 5, 2011

Related U.S. Application Data
(60) Provisional application No. 61/390,921, filed on Oct. 7, 2010.

(51) Int. Cl. A61K 9/50; B01J 13/04; A61K 38/26; A61P 3/10; A61K 38/02; A61K 38/28

(52) U.S. Cl. 424/491; 424/490; 424/499; 514/1.1; 514/5.9; 514/11.7; 427/213.31

(57) ABSTRACT

Disclosed herein are a class of small molecules, referred to as Small Peptide Sequences (SPS), that can stabilize biomolecules, particles containing the SPS and biomolecules, and compositions and methods of making the particles. The SPS are composed solely of amino acids common to humans and too small to trigger an immunological response (typically less than seven amino acids in total) and which will self assemble into particles sufficiently small to stay in liquid suspension at a first pH, typically a non-physiological pH, but which dissociate, releasing the biomolecule entrapped therein into solution, at a second pH, typically a physiological pH. The particles contain a SPS and a biomolecule, wherein the biomolecule is entrapped with the particle, immobilized on the surface of the particle, or combinations thereof. The particle releases the biomolecule upon contact with physiological fluids.

### CROSS REFERENCE TO RELATED APPLICATIONS

This application claims priority to U.S. Provisional Application Ser. No. 61/390,921 filed Oct. 7, 2010, the contents of which are herein incorporated by reference in their entirety.

### FIELD OF THE INVENTION

This invention is generally in the field of methods and reagents for stabilization of proteins and other bioactive molecules, such as proteins and nucleic acids, in aqueous solution or suspension.

### BACKGROUND OF THE INVENTION

Most biotechnology products, including peptides and proteins such as enzymes, antibodies, and antigens/vaccines, as well as nucleic acids such as RNA and DNA, are unstable in an aqueous suspension or solution. Many interact with each other when stored in the same vial, requiring patients to take multiple injections. Glucagon and glucose oxidase are but two of many examples of peptides which are unstable in aqueous solution, while pramlintide and insulin are an example of two drugs that should be co-administered but cannot be stored in the same solution.

It is an object of the present invention to provide materials, and methods of use thereof, to stabilize biomolecules such as proteins and nucleic acids, in an aqueous environment.

### SUMMARY OF THE INVENTION

A class of small molecules, referred to as Small Peptide Sequences (SPS), can be synthesized that are composed solely of amino acids common to humans and too small to trigger an immunological response (typically less than seven amino acids in total) and which will self assemble into particles sufficiently small to stay in liquid suspension at a first pH, typically a non-physiological pH, but which dissociate, releasing the biomolecule into solution, at a second pH, typically a physiological pH.

The Small Peptide Sequence (SPS) is soluble in a solvent, such as water or an aqueous medium. The biomolecule to be stabilized is added to the solution of the SPS. The SPS weakly attracts the biomolecule in solution, typically by a non-covalent interaction, such as hydrogen bonding or electrostatic interactions. As the pH is increased, the SPSs will self-assemble to form particles, entrapping the biomolecule within the particle and/or immobilizing it on the particle. The pH at which self-assembly occurs is dependent on the chemical composition of the SPS. However, the pH at which self-assembly occurs is typically close to the isoelectric point of the SPS. Once the SPSs self-assemble, the particles can be isolated, for example though filtration, dried, and stored for extended periods of time. For administration, the particles are resuspended in an appropriate solvent and administered to a subject. As the particles come into contact with a medium having a higher pH, such as physiological pH (e.g., 7-7.4), the particles dissociate releasing the entrapped or immobilized biomolecule back into solution, where it can be absorbed by the body.

The pH range at which the SPS forms complexes or particles can be controlled by the selection of amino acids in the sequence of the SPS. For example, poly-acidic amino acids such as aspartic acid (Asp) and glutamic acid (GLU) produce an SPS that self assembles below neutral pH and polybasic amino acids such as histidine (His), arginine (Arg) and lysine (Lys) produce an SPS that self assembles above neutral pH.

The choice of the amino acid also determines the tendency for the SPS to self assemble into small and uniform particles. Amino acids with dense electron centers such as amino acids containing a cyclic core, such as tryptophan, phenylalanine, tyrosine and proline, rotationally self-stack as they self assemble and tend to produce small, uniform particles. However, other peptide sequences can also precipitate out of solution at a particular pH and also attract an active pharmaceutical ingredient, (such as a therapeutic peptide, hormone, etc.), incorporating the active pharmaceutical ingredient and stabilizing it.

### DETAILED DESCRIPTION OF THE INVENTION

### I. Compositions

A. SPS

Self-assembling protein materials have been known for many decades. One of the best known self-assembling encapsulation systems is the amino acid polymer self-assembling microcapsules first pioneered by Dr. Sidney Fox (Molecular Evolution and the Origin of Life). The initial experiments were with “proteinoids”, a “linear thermal condensation polymer of a mixture of naturally occurring amino acids”, an undefined mixture of protein materials that lacked definition and reproducibility, although a method for delivery of drugs encapsulated in proteins was developed as described in U.S. Pat. No. 4,925,673 by Steiner, et al.

Recognizing the problems inherent in obtaining FDA approval of an undefined mixture such as proteins, diketopiperazine (cyclic amino di-amino acids) formulations were developed for purification or delivery of drugs, as described in U.S. Pat. No. 6,444,226 by Steiner, et al. Peptide or proteins were incorporated into a diketopiperazine to facilitate removal of one or more impurities. Formulations and methods were also described for the transport of active agents across biological membranes. More recently, self-assembling peptides have been described in U.S. published patent application No. 20050287186 by Ellis-Behnke, et al.

These systems all have drawbacks, since the materials are either difficult to characterize (proteinoids), raise FDA issues (proteins, diketopiperazines) or are complex (Ellis-Behnke)

In contrast, the SPSs described herein are small linear peptides. The peptides general contain less than seven amino acids. Peptides containing less than seven amino acids are less likely to cause an immunological response and are cheaper to manufacture. However, peptides containing more than 7 amino acids can also be used. Suitable peptides include di-, tri-, tetra-, penta-, hexa-, and septapeptides. In one embodiment, the peptide is a tripeptide.

The peptide can also interact with biomolecules, such as proteins or nucleic acid, to stabilize the biomolecule. The interactions between the peptide and the protein or nucleic acid to be stabilized are typically non-covalent interactions including, but not limited to, hydrogen bonding and electrostatic interactions. In one embodiment, the non-covalent interaction is hydrogen bonding. The selection and/or number of amino acids in the SPS can be varied to optimize the interaction between the SPS and the protein or nucleic acid to be stabilized.

In one embodiment, the SPSs are constructed of amino acids which have the ability to interact with each other to “stack” and form particles. This stacking function is pH dependent. The amino acids are selected to “stack” at a first, non-physiological pH, and “unstack” or dissociate at a second, physiological pH (e.g., 7-7.4), thereby releasing any biomolecules entrapped in, or immobilized on, the self-assembling particles.

A class of small molecules, referred to as Small Peptide Sequences, (SPS), can be synthesized that are composed solely of amino acids common to humans and too small to trigger an immunological response (typically less than seven amino acids in total) and which will self assemble into particles sufficiently small to stay in liquid suspension. The Small Peptide Sequence (SPS) is insoluble in a first pH range and soluble at a second pH range, typically physiological pH. The SPS weakly attracts a particular peptide or protein or nucleic acid molecule in solution by a force such as hydrogen bonding, causing the other molecule to precipitate out of solution and become associated with or entrapped within the SPS as it self-assembles to form particles at a first non-physiological pH. At or near physiological pH, e.g., 7-7.4, the particles formed of the SPS and the protein or nucleic acid to be stabilized dissociate and the protein or nucleic acid is released back into solution where it can be absorbed by the body.

The pH range at which the SPS self-assembles can be controlled by the selection of amino acids in the sequence of the SPS. For example, poly-acidic amino acids such as aspartic acid (Asp) and glutamic acid (GLU) produce an SPS that self assembles below neutral pH and polybasic amino acids such as histidine (His), arginine (Arg) and lysine (Lys) produce an SPS that self assembles above neutral pH. The choice of the amino acid also determines the tendency for the SPS to self assemble into small and uniform particles. Amino acids with dense electron centers such as amino acids containing a cyclic core, such as tryptophan, phenylalanine, tyrosine and proline, rotationally self-stack as they self assemble and tend to produce small and uniform particles.

B. Molecules to be Stabilized

Therapeutic, prophylactic or diagnostic molecules to be stabilized include any polymer or large organic molecules, most preferably peptides and proteins. Generally speaking, any form of drug can be entrapped. Examples include synthetic inorganic and organic compounds, proteins and peptides, polysaccharides and other sugars, lipids, and nucleic acid sequences having therapeutic, prophylactic or diagnostic activities. Proteins are defined as consisting of 100 amino acid residues or more; peptide are less than 100 amino acid residues. Unless otherwise stated, the term protein refers to both proteins and peptides. The agents to be incorporated can have a variety of biological activities, such as vasoactive agents, neuroactive agents, hormones, anticoagulants, immunomodulating agents, cytotoxic agents, antibiotics, antivirals, antisense, antigens, and antibodies. In some instances, the proteins may be antibodies or antigens

Preferred peptides and proteins include hormones, cytokines and other immunomodulatory peptides, and antigens/vaccines. In a preferred embodiment, the active agent is a stabilized form of insulin which has been purified to remove zinc. In another preferred embodiment, the active agent is glucagon. In another embodiment, the active agent is the enzyme glucose oxidase. The active agent, or drug, can be an antigen, where the molecule is intended to elicit a protective immune response. In these cases, it may also be useful to administer the drug in combination with an adjuvant, to increase the immune response to the antigen.

Any genes that would be useful in replacing or supplementing a desired function, or achieving a desired effect such as the inhibition of tumor growth, could be entrapped in the SPS particles. As used herein, a “gene” is an isolated nucleic acid molecule of greater than thirty nucleotides, preferably one hundred nucleotides or more, in length. Examples of genes which replace or supplement function include the genes encoding missing enzymes such as adenosine deaminase (ADA) which has been used in clinical trials to treat ADA deficiency and cofactors such as insulin and coagulation factor VIII. Genes which effect regulation can also be administered, alone or in combination with a gene supplementing or replacing a specific function. For example, a gene encoding a protein which suppresses expression of a particular protein-encoding gene, or vice versa, which induces expresses of a protein-encoding gene, can be administered in the matrix. Examples of genes which are useful in stimulation of the immune response include viral antigens and tumor antigens, as well as cytokines (tumor necrosis factor) and inducers of cytokines (endotoxin), and various pharmacological agents. Other nucleic acid sequences that can be utilized include antisense molecules which bind to complementary DNA to inhibit transcription, ribozyme molecules, and external guide sequences used to target cleavage by RNAase P.

As used herein, vectors are agents that transport the gene into targeted cells and include a promoter yielding expression of the gene in the cells into which it is delivered. Promoters can be general promoters, yielding expression in a variety of mammalian cells, or cell specific, or even nuclear versus cytoplasmic specific. These are known to those skilled in the art and can be constructed using standard molecular biology protocols. Vectors increasing penetration, such as lipids, liposomes, lipid conjugate forming molecules, surfactants, and other membrane permeability enhancing agents are commercially available and can be delivered with the nucleic acid.

Imaging agents including metals, radioactive isotopes, radioopaque agents, fluorescent dyes, and radiolucent agents also can be incorporated. Examples of radioisotopes and radioopaque agents include gallium, technetium, indium, strontium, iodine, barium, and phosphorus.

C. Excipients

The formulations are typically prepared in an aqueous solution, which may be saline or phosphate buffered saline. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Solubilizing agents are included to promote rapid dissolution in aqueous media. Suitable solubilizing agents include wetting agents such as polysorbates, glycerin and poloxamers, non-ionic and ionic surfactants, food acids and bases (e.g. sodium bicarbonate), and alcohols, and buffer salts for pH control.

Stabilizers are used to inhibit or retard drug decomposition reactions which include, by way of example, oxidative reactions. A number of stabilizers may be used. Suitable stabilizers include polysaccharides, such as cellulose and cellulose derivatives, and simple alcohols, such as glycerol; bacteriostatic agents such as phenol, m-cresol and methylparaben; isotonic agents, such as sodium chloride, glycerol, and glucose; lecithins, such as example natural lecithins (e.g. egg yolk lecithin or soya bean lecithin) and synthetic or semisynthetic lecithins (e.g. dimyristoylphosphatidylcholine, dipalmitoylphosphatidylcholine or distearoyl-phosphatidylcholine; phosphatidic acids; phosphatidylethanolamines; phosphatidylserines such as distearoyl-phosphatidylserine, dipalmitoylphosphatidylserine and diarachidoylphospahtidylserine; phosphatidylglycerols; phosphatidylinositols; cardiolipins; sphingomyelins. In one example, the stabilizer may be a combination of glycerol, bacteriostatic agents and isotonic agents.

### II. Methods of Stabilization

Applications

To enhance formulation stability, glucose oxidase at a concentration of 50 unit/ml in an insulin formulation such as glargine at a concentration of 100 units/ml may be precipitated using an SPS at a concentration of 265 mg/ml. At pH 4 where glargine is soluble, the SPS is insoluble, keeping its cargo separate from the glargine in solution. Post injection, the pH of the internal milieu increases to pH ˜7-7.4 of the injection bolus, releasing the glucose oxidase intact and the glargine precipitates.

Glucagon stability is increased by precipitating with SPS at low pH, for example about 3.8-4.8 at a concentration of 1 mg/ml. The particle suspension is stabilized at room temperature. No reconstitution is necessary. Upon injection, the precipitate dissociates to release glucagon into the systemic circulation

### III. Kits

Kits containing the ingredients to form the stabilized particles or the stabilized particles themselves are described herein. In one embodiment, the kit contains particles containing the SPS and the stabilized biomolecule. The particles are typically in a container, such as a glass or plastic jar. Prior to use, the particles can be resuspended in a pharmaceutically acceptable solvent, optionally containing one or more pharmaceutically acceptable excipients. The suspension can be taken up in a syringe or other medical device for administration. The kit can contain the medical device or it can be supplied by the end user. The kit can contain instructions for preparing the particles and/or resuspending the particles for administration.

In another embodiment, the kit contains the SPS in one container and the biomolecule in a second container, along with instructions for preparing and administering the stabilized particles.

The present invention will be further understood by reference to the following non-limiting examples.

### EXAMPLES

### Example 1

Synthesis of SPS and Precipitation of Glucagon

The following small peptide sequences were synthesized:
1. H-Aspartic Acid-Tryptophan-Aspartic Acid —OH
2. H-Glutamic acid-Tryptophan-Glutamic acid —OH
3. H-Aspartic Acid-Tryptophan-Glutamic acid —OH

These sequences are soluble at very low pH and form microparticles that trap peptides in solution within the particles. They remain intact at pH 3-5 in the vicinity of their isoelectric point. Solubility increases in the physiological pH range.

Glucagon was entrapped very efficiently, (more than 60% of the glucagon remained in the particles) using H-Aspartic Acid-Tryptophan-Aspartic Acid —OH, which stabilized it in aqueous solution and released the glucagon intact when the pH of the solution was brought to neutral.

Glucose oxidase was similarly stabilized and precipitated by H-Aspartic Acid-Tryptophan-Aspartic Acid —OH, in aqueous solution, which allowed it to be stored in aqueous solution along with insulin glargine at a pH of 4. The glucose oxidase was released intact when the pH of the solution was brought to neutral.

A light microscopy image was taken of SPS in suspension in the mildly acidic range of pH about 5. The light microscopy image of ASP-TRP-ASP shows a size range of about 300-900 μm.

We claim:

1. Particles comprising a small peptide sequence and a biomolecule, wherein the biomolecule is entrapped with the particle, immobilized on the surface of the particle, or combinations thereof, wherein the particle releases the biomolecule upon contact with physiological fluids.

2. The particles of claim 1, wherein the biomolecule is a peptide, polypeptide, protein, nucleic acid, or combinations thereof.

3. The particles of claim 2, wherein the biomolecule is a polypeptide.

4. The particles of claim 3, wherein the polypeptide is insulin.

5. The particles of claim 4, wherein the polypeptide is glucagon.

6. The particles of claim 1, wherein the SPS comprises an amino acid having a dense electron center.

7. The particles of claim 6, wherein the amino acid is selected from the group consisting of tryptophan, phenylalanine, tyrosine, and proline.

8. The particles of claim 1, wherein the small peptide sequence contains seven amino acids or less.

9. The particles of claim 8, wherein the peptide is a tripeptide.

10. The particles of claim 9, wherein the tripeptide is Asp-Trp-Asp.

11. The particles of claim 9, wherein the tripeptide is Glu-Trp-Glu.

12. The particles of claim 9, wherein the tripeptide is Asp-Trp-Glu.

13. The particles of claim 1, wherein the particles are formed by dissolving the SPS and the biomolecule in a solvent and raising the pH until the SPSs self-assemble entrapping and/or immobilizing the biomolecule.

14. The particles of claim 13, wherein the pH at which the SPS self-assemble is about the isoelectric point of the SPS.

15. The particles of claim 14, wherein the isoelectric point is less than 7.

16. The particles of claim 14, wherein the isoelectric point is greater than 7.

17. The particles of claim 1, wherein the pH of the physiological fluids is from about 6.8 to about 7.4.

18. A method for making the particles of claim 1, comprising
(a) dissolving a small peptide sequence (SPS) and biomolecule to be stabilized in a solvent; and
(b) raising the pH to the point where the SPS self-assembles to form particles having entrapped within, immobilized thereon, or combinations thereof the biomolecules to be stabilized

19. The method of claim 18, wherein the particles are isolated from the solvent.

20. The method of claim 19, wherein the particles are dried to remove the solvent.

21. A pharmaceutical composition comprising the particles of claim 1 and a physiologically acceptable carrier.

22. The composition of claim 21, further comprising one or more pharmaceutically acceptable excipients.
